(19) United States
(12) Patent Application Publication
(10) Pub. No.: US 2025/0352504 A1
(43) Pub. Date: Nov. 20, 2025

(54) LEVOCARNITINE AND TRIMETAZIDINE BILAYER TABLET AND PREPARATION METHOD THEREOF

(71) Applicant: Changzhou High-tech Industrial Development Zone 3D Industrial Tech. Research Institute Co., Ltd., Changzhou City (CN)

(72) Inventors: Jie Zong, Changzhou City (CN); Lu Zhang, Changzhou City (CN); Chengjie Xue, Changzhou City (CN); Yingying Li, Changzhou City (CN); Rong Xu, Changzhou City (CN)

(21) Appl. No.: 18/667,827

(22) Filed: May 17, 2024

(51) Int. Cl. A61K 31/205; A61K 9/20; A61K 9/24; A61K 31/495

(52) U.S. Cl. CPC A61K 31/205; A61K 9/2013; A61K 9/2018; A61K 9/2027; A61K 9/2054; A61K 9/209; A61K 9/2095; A61K 31/495

(57) ABSTRACT

Disclosed is a levocarnitine and trimetazidine bilayer tablet. The present disclosure adopts a bilayer structure of the levocarnitine and trimetazidine dihydrochloride, and a contact area between the levocarnitine layer and the air can be greatly reduced by covering one side of a surface of the levocarnitine layer with the trimetazidine dihydrochloride layer, such that the problem of hygroscopicity of the levocarnitine layer can be effectively inhibited, and the effective amount of the drug is increased. Moreover, the preparation process in the present disclosure is easy to operate and suitable for actual production and use, as well as is capable of solving the problem of content uniformity well, and the stability of the bilayer tablet is effectively improved.

### TECHNICAL FIELD

The present disclosure belongs to the field of medical technology, and particularly relates to a levocarnitine and trimetazidine bilayer tablet and a preparation method thereof.

### BACKGROUND

Levocarnitine, also known as (3R)-3-hydroxy-4-(trimethylammonio) butanoate, is white or off-white crystalline powder with strong hygroscopicity. It is readily soluble in water or ethanol, almost insoluble in acetone or ether, and easily soluble in formic acid. Levocarnitine is clinically used for the prevention and treatment of levocarnitine deficiency, and suitable for a series of complications caused by secondary carnitine deficiency in patients with chronic renal failure and long-term hemodialysis. Clinical manifestations include cardiomyopathy, skeletal myopathy, arrhythmia, hyperlipidemia, hypotension, muscle spasms during dialysis, and the like. Structure of the levocarnitine is as follows:

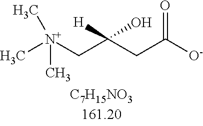

Trimetazidine dihydrochloride, also known as 1-(2,3,4-Trimethoxybenzyl) piperazine dihydrochloride, is white or off-white and odorless crystalline powder. It is highly soluble in water, easily soluble in acetic acid, soluble in methanol, slightly soluble in ethanol, and almost insoluble in ether. It is highly soluble in 0.1 mol/L hydrochloric acid solution or 0.1 mol/L sodium hydroxide solution. It is clinically used as an adjunctive therapy in adults for symptomatic treatment of patients with stable angina pectoris poorly controlled or intolerant to first-line antianginal therapy.

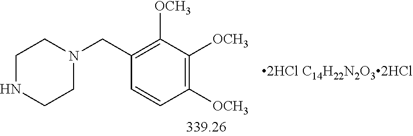 [SMILES: COc1ccc(CN2CCNCC2)c(OC)c1OC]

The Chinese Patent CN102058888A discloses a combination of levocarnitine and trimetazidine dihydrochloride in different proportions for the prevention or treatment of diseases caused by abnormal energy metabolism, and it is of great significance to comprehensively balancing the metabolism of fatty acid and glucose, optimizing the productivity of fatty acid and glucose, effectively treating mitochondrial dysfunction, reducing side effects of trimetazidine, and promoting physiological and pathological recovery. The patent further a prescription process for preparing a compound tablet by mixing and granulating levocarnitine, trimetazidine dihydrochloride with starch and lactose. However, the prescription process has problems such as high content of impurities.

The Chinese Patent CN107693521A discloses a method of mixing levocarnitine, trimetazidine dihydrochloride and anti-hygroscopic excipients in a specific proportion and compressed into tablets to achieve significant anti-hygroscopicity. Further, in the patent, a main drug levocarnitine, and trimetazidine dihydrochloride are granulated separately, and two granules obtained thereby are then mixed in specific proportions and then compressed to obtain tablets, so as to solve the problem of uneven content of the main drug in the tablets. The preparation process of compound levocarnitine tablets disclosed in the patent involves: mixing levocarnitine active pharmaceutical ingredient, copovidone, microcrystalline cellulose, and lactose evenly, adding 90% ethanol and granulating, and drying the whole granules to obtain levocarnitine granules; mixing trimetazidine dihydrochloride, copovidone, microcrystalline cellulose, and lactose evenly, adding 90% ethanol and granulating, and drying the whole granules to obtain trimetazidine dihydrochloride granules; and mixing talcum powder, magnesium stearate, the levocarnitine granules and the trimetazidine dihydrochloride granules evenly and compressing to obtain tablets. Although the process solves problem of uneven drug content of the main drug to some extent, significantly increases the use of excipients, resulting in an increase in costs and failing to improve the stability thereof effectively.

In the prior art, lactose, as an excipient, contains impurities such as sucrose, furfural, formic acid, and acetic acid. Another excipient copovidone or crospovidone contain residual peroxides. Microcrystalline cellulose, as an excipient, contains reactive impurities including sucrose, formaldehyde, and the like. An active pharmaceutical ingredient, trimetazidine dihydrochloride, contain piperidine ring and a secondary amine structure, which may undergo Maillard reaction with sucrose, react with formic acid to form acylation impurities, react with peroxide impurities to form N-oxides, and react with formaldehyde to form impurities.

Reactions between drugs and pharmaceutical excipients and their active impurities are listed below:

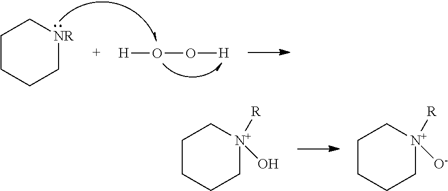

### Formula 1: Hydrogen Peroxide Impurities Generate N

### Addition Reaction of Oxides and Amines

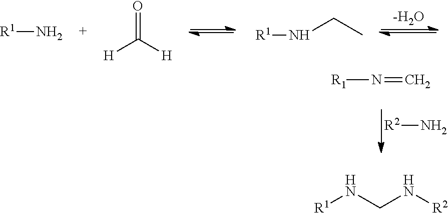

### Formula 2: Reaction of Amines and Formaldehyde

In summary, none of the prescription processes disclosed in the prior art solves the problem of stability of tablets of levocarnitine and trimetazidine. In order to control impurities in the tablets of levocarnitine and trimetazidine, and improve their quality, it is urgent to develop a dosage form and preparation process with reasonable design, controllable quality and good stability.

### SUMMARY

An objective of the present disclosure is to provide a compound pharmaceutical preparation containing levocarnitine and trimetazidine dihydrochloride that meets the requirements in terms of stability, uniformity, and the like. According to the present disclosure, levocarnitine and trimetazidine dihydrochloride are prepared into a bilayer tablet, enabling their active ingredients to be distributed in different layers, moreover, an appropriate amount of excipients are added to one or both of the layers, which can completely overcome the above problems of stability and uniformity. The present disclosure is implemented based on the aforesaid discovery.

To this end, a first aspect of the present disclosure provides a levocarnitine and trimetazidine bilayer tablet, including:
a levocarnitine layer composed of a levocarnitine composition, and
a trimetazidine layer composed of a trimetazidine dihydrochloride composition.

A levocarnitine and trimetazidine bilayer tablet according to any one of the first aspect of the present disclosure, where a weight ratio of the levocarnitine and the trimetazidine dihydrochloride included in the bilayer tablet is 100-200:1, the weight ratio of the same in one embodiment is 200:1, the weight ratio of the same in another embodiment is 150:1, and the weight ratio of the same in another embodiment is 190:1. In a preferred embodiment, the weight ratio of the same is 190:1.

The levocarnitine and trimetazidine bilayer tablet according to any one of the first aspect of the present disclosure, where the levocarnitine composition includes levocarnitine, an adhesive, a filler, a glidant, and a lubricant; preferably, 400-600 parts by weight of the levocarnitine, 15-25 parts by weight of the adhesive, 150-200 parts by weight of the filler, 15-25 parts by weight of the glidant, and 1-5 parts by weight of the lubricant, and in a preferred embodiment, 500 parts by weight of levocarnitine, 20 parts by weight of the adhesive, 170 parts by weight of the filler, 22 parts by weight of the glidant, and 3 parts by weight of the lubricant are selected; and
the levocarnitine and trimetazidine bilayer tablet according to any one of the first aspect of the present disclosure, where the trimetazidine dihydrochloride composition includes trimetazidine dihydrochloride, a filler A, and a lubricant; preferably, 3-6 parts by weight of the levocarnitine, 60-80 parts by weight of the filler A, and 0.1-0.5 parts by weight of the lubricant; and in a preferred embodiment, 4 parts by weight of the levocarnitine, 68 parts by weight of the filler A, and 0.3 parts by weight of the lubricant.

The levocarnitine and trimetazidine bilayer tablet according to any one of the first aspect of the present disclosure, in order to effectively distinguish the levocarnitine layer and the trimetazidine layer, an appropriate amount of colorant, such as red iron oxide, can be added in any one of them therein.

The levocarnitine and trimetazidine bilayer tablet according to any one of the first aspect of the present disclosure, the present disclosure provides a preparation method of the levocarnitine composition, including the following steps: mixing the levocarnitine, the adhesive and the filler together, adding absolute ethanol for granulating, performing drying and dry granulation to obtain dry granules, mixing the dry granules with talcum powder and magnesium stearate in sequence to obtain the levocarnitine composition; and
the present disclosure further provides a preparation method of the trimetazidine dihydrochloride composition, including the following steps: mixing the trimetazidine dihydrochloride and the filler A together to obtain material, mixing the material with the lubricant to obtain the trimetazidine dihydrochloride composition.

The levocarnitine and trimetazidine bilayer tablet according to any one of the first aspect of the present disclosure, where povidone is used as the adhesive, and in order to achieve better effects, povidone (K30) can be selected as the povidone; microcrystalline cellulose is used as the filler, and in order to achieve better effects, microcrystalline cellulose (PH101) can be selected as the microcrystalline cellulose; mannitol is used as the filler A, and in order to achieve better effects, mannitol (200SD) can be selected as the mannitol; and talcum powder is used as the glidant, and magnesium stearate is used as the lubricant.

A second aspect of the present disclosure provides a preparation method of the levocarnitine and trimetazidine bilayer tablet according to any one of the first aspect of the present disclosure, including: putting the levocarnitine composition into a first-layer hopper of a bilayer tablet press, putting the trimetazidine dihydrochloride composition into a second-layer hopper of the bilayer tablet press, and setting main pressures and main pressure thicknesses of a first gear and a second gear of the bilayer tablet press respectively to press and obtain the bilayer tablet.

The present disclosure has the following beneficial effects:
(1) The present disclosure adopts a bilayer structure of the levocarnitine and trimetazidine dihydrochloride, and a contact area between the levocarnitine layer and the air can be greatly reduced by covering one side of a surface of the levocarnitine layer with the trimetazidine dihydrochloride layer, such that the problem of hygroscopicity of the levocarnitine layer can be effectively inhibited.
(2) The present disclosure adopts a bilayer structure of the levocarnitine and trimetazidine dihydrochloride, such that the effective amount of the drug is increased. Under the condition that the mixing is performed by using the prior art, the effect of uniform mixing cannot be achieved. However, the preparation process in the present disclosure is easy to operate and suitable for actual production and use, as well as is capable of solving the problem of content uniformity well.
(3) The content of oxidized impurities and total impurities of the levocarnitine and trimetazidine bilayer tablet prepared according to the present disclosure are significantly lower than those of the tablet prepared in the prior art, such that the stability of the bilayer tablet is effectively improved.

### DETAILED DESCRIPTIONS OF THE EMBODIMENTS

The present disclosure will be further described below through specific embodiments and experimental examples. However, it should be understood that these embodiments and experimental examples are merely used for more detailed description, and should not be construed as limiting the present disclosure in any form.

The present disclosure gives a general and/or specific description of materials and test methods used in the tests. Although many materials and procedures of operation used for achieving the objectives of the present disclosure are well known in the art, the present disclosure will still describe in detail them herein. It will be apparent to those skilled in the art that the materials and procedures of operation used in the present disclosure, unless otherwise specified, are well known in the art.

### Example 1

### Preparation of Levocarnitine and Trimetazidine Bilayer Tablets

### Components

| Levocarnitine layer | | Trimetazidine layer | |
|---|---|---|---|
| Ingredient | Weight(mg) | Ingredient | Weight(mg) |
| Levocarnitine | 570 | Trimetazidine | 3 |
| | | dihydrochloride | |
| Povidone | 20 | Mannitol | 68 |
| Cellulose | 170 | Magnesium stearate | 0.3 |
| Talcum powder | 22 | | |
| Magnesium stearate | 3 | | |

### Preparation Method:

step (1) preparation of a levocarnitine composition: levocarnitine, povidone and microcrystalline cellulose were premixed by using a high-speed shearing wet granulator, absolute ethanol was added for granulating, wet levocarnitine composition granules were then obtained, the wet levocarnitine composition granules were dried on a fluidized bed, with a drying endpoint of LOD being less than 2.5%, dry granulation was performed to obtain dry levocarnitine composition granules, and the levocarnitine composition granules were mixed with talcum powder and magnesium stearate in sequence to obtain the levocarnitine composition;
step (2) preparation of a trimetazidine dihydrochloride composition: trimetazidine dihydrochloride and mannitol were mixed by using the high-speed shearing wet granulator to obtain granules, the granules were then mixed with magnesium stearate to obtain the trimetazidine dihydrochloride composition; and
step (3) preparation of a bilayer tablet: the levocarnitine composition was put into a first-layer hopper of a bilayer tablet press, and the trimetazidine dihydrochloride composition was put into a second-layer hopper of the bilayer tablet press, and main pressures and main pressure thicknesses of a first gear and a second gear of the bilayer tablet press were set respectively to press and obtain the bilayer tablet.

In the preparation process, an appropriate amount of colorant can be added to the steps (1) or (2) to distinguish different layers, without affecting other properties of the bilayer tablet.

### Example 2

### Preparation of Levocarnitine and Trimetazidine Bilayer Tablets

### Components

| Levocarnitine layer | | Trimetazidine layer | |
|---|---|---|---|
| Components | Weight(mg) | Components | Weight(mg) |
| Levocarnitine | 600 | Trimetazidine | 3 |
| | | dihydrochloride | |
| Povidone | 25 | Mannitol | 60 |
| Cellulose | 200 | Magnesium stearate | 0.1 |
| Talcum powder | 25 | | |
| Magnesium stearate | 5 | | |

### Preparation Method:

step (1) preparation of a levocarnitine composition: levocarnitine, povidone and microcrystalline cellulose were premixed by using a high-speed shearing wet granulator, absolute ethanol was added for granulating, wet levocarnitine composition granules were then obtained, the wet levocarnitine composition granules were dried on a fluidized bed, with a drying endpoint of LOD being less than 2.5%, dry granulation was performed to obtain dry levocarnitine composition granules, and the levocarnitine composition granules were mixed with talcum powder and magnesium stearate in sequence to obtain the levocarnitine composition;
step (2) preparation of a trimetazidine dihydrochloride composition: trimetazidine dihydrochloride and mannitol were mixed by using the high-speed shearing wet granulator to obtain granules, the granules were then mixed with magnesium stearate to obtain the trimetazidine dihydrochloride composition; and
step (3) preparation of a bilayer tablet: the levocarnitine composition was put into a first-layer hopper of a bilayer tablet press, and the trimetazidine dihydrochloride composition was put into a second-layer hopper of the bilayer tablet press, and main pressures and main pressure thicknesses of a first gear and a second gear of the bilayer tablet press were set respectively to press and obtain the bilayer tablet.

### Example 3

### Preparation of Levocarnitine and Trimetazidine Bilayer Tablets

### Components

| Levocarnitine layer | | Trimetazidine layer | |
|---|---|---|---|
| Components | Weight(mg) | Components | Weight(mg) |
| Levocarnitine | 600 | Trimetazidine | 4 |
| | | dihydrochloride | |
| Povidone | 15 | Mannitol | 80 |
| Cellulose | 150 | Magnesium stearate | 0.5 |
| Talcum powder | 15 | | |
| Magnesium stearate | 1 | | |

### Preparation Method:

step (1) preparation of a levocarnitine composition: levocarnitine, povidone and microcrystalline cellulose were premixed by using a high-speed shearing wet granulator, absolute ethanol was added for granulating, wet levocarnitine composition granules were then obtained, the wet levocarnitine composition granules were dried on a fluidized bed, with a drying endpoint of LOD being less than 2.5%, dry granulation was performed to obtain dry levocarnitine composition granules, and the levocarnitine composition granules were mixed with talcum powder and magnesium stearate in sequence to obtain the levocarnitine composition;
step (2) preparation of a trimetazidine dihydrochloride composition: trimetazidine dihydrochloride and mannitol were mixed by using the high-speed shearing wet granulator to obtain granules, the granules were then mixed with magnesium stearate to obtain the trimetazidine dihydrochloride composition; and
step (3) preparation of a bilayer tablet: the levocarnitine composition was put into a first-layer hopper of a bilayer tablet press, and the trimetazidine dihydrochloride composition was put into a second-layer hopper of the bilayer tablet press, and main pressures and main pressure thicknesses of a first gear and a second gear of the bilayer tablet press were set respectively to press and obtain the bilayer tablet.

### Example 4

It is basically the same as Example 1, except that povidone (K30) was selected as the povidone, microcrystalline cellulose (PH101) was selected as the microcrystalline cellulose, and mannitol (200SD) was selected as the mannitol.

### Comparative Example 1

Raw materials were taken according to the component ratio in Example 1, and single-layer tablets were prepared by the following method:

Preparation Steps were as Follows:
step (1) preparation of granules: levocarnitine, trimetazidine dihydrochloride, povidone, microcrystalline cellulose and mannitol were premixed by using the high-speed shearing wet granulator, and an absolute ethanol solution was added for granulation to obtain wet granules;
step (2) drying and granulation: the wet granules were dried on the fluidized bed, with a drying endpoint of LOD being less than 2.5%, dry granulation was performed to obtain dry granules;
step (3) mixing: the dry granules were mixed with talcum powder and magnesium stearate to obtain total mixed granules; and
step (4) tablet pressing: the total mixed granules were added to a hopper of a tablet press, a filling depth was adjusted to achieve a target piece weight, a main pressure interval was adjusted to achieve a target hardness, and compound levocarnitine and trimetazidine dihydrochloride tablets were obtained.

### Comparative Example 2

The compound levocarnitine tablets prepared in Example 4 in the Chinese Patent CN107693521A were used as Comparative Example 2.

Tests were performed on Examples 1-4, and Comparative Example 1-2:

### Hygroscopicity Investigation:

With reference to range of general temperature and humidity of GMP workshops and production time of the workshops, the hygroscopicity (measured by weight gain percentage) of the finished tablets in each example under the conditions that a temperature was 22° C., a humidity was 65%, and RH exposure was 2.5 h. Results of the weight gain percentage were as follows:

As can be seen from the results that the weight gain percentages of Examples 1˜4 are significantly smaller than those of Comparative Examples 1-2, indicating that the hygroscopicity of Examples 1˜4 is less than that of Comparative Examples 1-2.

### Content Uniformity

For the pressed tablets, the content uniformity of the levocarnitine composition and the trimetazidine composition were measured in accordance with the Technical Guiding Principles for Research on Mixing Uniformity and In-process Dosage Unit Uniformity of Chemical Pharmaceutical Oral Solid Dosage.

| | Levocarnitine | Trimetazidine |
|---|---|---|
| Examples | RSD value/% | RSD value/% |
| Example 1 | 1.33 | 1.51 |
| Example 2 | 1.46 | 1.62 |
| Example 3 | 1.47 | 1.72 |
| Example 4 | 1.13 | 1.44 |
| Comparative Example 1 | 3.01 | 3.24 |
| Comparative Example 2 | 4.92 | 5.52 |

According to the Technical Guiding Principles for Research on Mixing Uniformity and In-process Dosage Unit Uniformity of Chemical Pharmaceutical Oral Solid Dosage, RSD≤6.0% is an acceptable range. Examples 1-4 and Comparative Examples 1-2 all meet the requirements for the content uniformity, but the Comparative Examples 1-2 are close to the limit of uniformity, and Examples 1-4 are smaller.

### Stability Investigation

Changes of substances related to trimetazidine (oxidized impurities, other largest single impurities, and total impurities) were taken as measurement index, and influencing factors were observed at 60° C. for 30 days, with the results as follows:

| Conditions of placement | | 0 day | 5 days | 10 days | 30 days |
|---|---|---|---|---|---|
| Example 1 | Oxidized | 0.05 | 0.10 | 0.12 | 0.21 |
| | impurities % | | | | |
| | Other largest | / | 0.14 | 0.15 | 0.17 |
| | single | | | | |
| | impurities % | | | | |
| | Total impurities % | 0.05 | 0.24 | 0.34 | 0.46 |
| Embodiment 2 | Oxidized | 0.06 | 0.11 | 0.14 | 0.25 |
| | impurities % | | | | |
| | Other largest | / | 0.15 | 0.12 | 0.19 |
| | single | | | | |
| | impurities % | | | | |
| | Total impurities % | 0.06 | 0.26 | 0.31 | 0.39 |
| Embodiment 3 | Oxidized | 0.05 | 0.10 | 0.16 | 0.22 |
| | impurities % | | | | |
| | Other largest | / | 0.18 | 0.21 | 0.23 |
| | single | | | | |
| | impurities % | | | | |
| | Total impurities % | 0.05 | 0.28 | 0.42 | 0.48 |
| Embodiment 4 | Oxidized | 0.05 | 0.09 | 0.12 | 0.18 |
| | impurities % | | | | |
| | Other largest | / | 0.11 | 0.13 | 0.13 |
| | single | | | | |
| | impurities % | | | | |
| | Total impurities | 0.05 | 0.20 | 0.29 | 0.33 |
| Comparative | Oxidized | 0.08 | 0.23 | 0.33 | 0.41 |
| Example 1 | impurities % | | | | |
| | Other largest | / | 0.20 | 0.32 | 0.31 |
| | single | | | | |
| | impurities % | | | | |
| | Total impurities % | 0.08 | 0.65 | 0.93 | 1.2 |
| Comparative | Oxidized | 0.23 | 0.45 | 0.68 | 0.77 |
| Example 2 | impurities % | | | | |
| | Other largest | 0.09 | 0.53 | 0.71 | 0.81 |
| | single | | | | |
| | impurities % | | | | |
| | Total impurities % | 0.37 | 2.68 | 2.96 | 3.52 |

The results show that Examples 1-4 are generally superior to Comparative Examples 1-2 in terms of stability, and Examples 1-4 show the minimum oxidized impurities, other largest single impurities, and total impurities.

After the preferred embodiments are described in details, those skilled in the art could clearly understand that various variations and modifications can be made without departing from the scope and spirit of the present disclosure, and any simple modifications, equivalent variations and modifications made to the above embodiments based on the technical essence of the present disclosure fall within the scope of the technical solutions of the present disclosure. Moreover, the present disclosure is not limited by the embodiments described in the specification.

What it claimed is:

1. A levocarnitine and trimetazidine bilayer tablet, comprising a levocarnitine layer composed of a levocarnitine composition, and a trimetazidine layer composed of a trimetazidine dihydrochloride composition; and a weight ratio of the levocarnitine and the trimetazidine dihydrochloride is 100-200:1.

2. The levocarnitine and trimetazidine bilayer tablet according to claim 1, wherein the levocarnitine composition comprises levocarnitine, an adhesive, a filler, a glidant, and a lubricant; and
the trimetazidine dihydrochloride composition comprises trimetazidine dihydrochloride, a filler A, and a lubricant.

3. The levocarnitine and trimetazidine bilayer tablet according to claim 2, wherein the levocarnitine composition comprises 400-600 parts by weight of the levocarnitine, 15-25 parts by weight of the adhesive, 150-200 parts by weight of the filler, 15-25 parts by weight of the glidant, and 1-5 parts by weight of the lubricant; and
the trimetazidine dihydrochloride composition comprises 3-6 parts by weight of the levocarnitine, 60-80 parts by weight of the filler A, and 0.1-0.5 parts by weight of the lubricant.

4. The levocarnitine and trimetazidine bilayer tablet according to claim 3, wherein the levocarnitine composition comprises 570 parts by weight of the levocarnitine, 20 parts by weight of the adhesive, 170 parts by weight of the filler, 22 parts by weight of the glidant, and 3 parts by weight of the lubricant; and
the trimetazidine dihydrochloride composition comprises 3 parts by weight of the levocarnitine, 68 parts by weight of the filler A, and 0.3 parts by weight of the lubricant.

5. The levocarnitine and trimetazidine bilayer tablet according to claim 1, wherein a preparation method of the levocarnitine composition comprises the following steps: mixing the levocarnitine, the adhesive and the filler together, adding absolute ethanol for granulating, performing drying and dry granulation to obtain dry granules, mixing the dry granules with talcum powder and magnesium stearate in sequence to obtain the levocarnitine composition; and
a preparation method of the trimetazidine dihydrochloride composition comprises the following steps: mixing the trimetazidine dihydrochloride and the filler A together to obtain material, mixing the material with the lubricant to obtain the trimetazidine dihydrochloride composition.

6. The levocarnitine and trimetazidine bilayer tablet according to claim 2, wherein povidone is used as the adhesive.

7. The levocarnitine and trimetazidine bilayer tablet according to claim 2, wherein microcrystalline cellulose is used as the filler, and mannitol is used as the filler A.

8. The levocarnitine and trimetazidine bilayer tablet according to claim 2, wherein talcum powder is used as the glidant.

9. The levocarnitine and trimetazidine bilayer tablet according to claim 2, wherein magnesium stearate is used as the lubricant.

10. A preparation method of the levocarnitine and trimetazidine bilayer tablet according to claim 1, comprising the following steps: putting the levocarnitine composition into a first-layer hopper of a bilayer tablet press, putting the trimetazidine dihydrochloride composition into a second-layer hopper of the bilayer tablet press, and setting main pressures and main pressure thicknesses of a first gear and a second gear of the bilayer tablet press respectively to press and obtain the bilayer tablet.
